(19) United States
(12) Patent Application Publication
(10) Pub. No.: US 2005/0131073 A1
(43) Pub. Date: Jun. 16, 2005

(54) Enantiomerically enriched 1-phenylethylamines

(76) Inventors: Bjorn Schlummer, Leverkusen (DE); Claus Dreisbach, Leichlingen (DE); Alain Cotte, Leverkusen (DE)

Correspondence Address: LANXESS CORPORATION, 111 RIDC PARK WEST DRIVE, PITTSBURGH, PA, 15275-1112, US

(21) Appl. No.: 10/910,942

(22) Filed: Aug. 4, 2004

(30) Foreign Application Priority Data
Aug. 7, 2003 (DE) 10336185.5

(51) Int. Cl. A61K 31/135

(52) U.S. Cl. 514/649; 564/336; 562/30

(57) ABSTRACT

The present invention relates to a process for preparing nitro-substituted, enantiomerically enriched 1-phenylethylamines, and to their use.

### BACKGROUND OF THE INVENTION

1. Field of the Invention

The present invention relates to a process for preparing nitro-substituted, enantiomerically enriched 1-phenylethylamines, and to their use.

2. Brief Description of the Prior Art

Nitro-substituted, enantiomerically enriched 1-phenylethylamines are important intermediates for the synthesis of active pharmaceutical ingredients, especially inosine-5′-monophosphate dehydrogenase (IMPDH) inhibitors (see also WO-A00/56331). In general, nitro-substituted 1-phenylethylamines can be prepared, for example, via the reductive amination of nitro-substituted acetophenones.

For example, F. Nerdel, H. Liebig, Liebigs Ann. d. Chemie 1954, 87, 221-222 disclose the preparation of 1-(3-nitrophenyl)ethylamine hydrochloride by reacting 3-nitroacetophenone with ammonium carbonate and formic acid at 190° C.

A. de Roocker, P. de Radzitzky, Bull. Soc. Chim. Belg 1963, 202-207 describe the preparation of 1-(3-nitrophenyl)ethylamine by reacting 3-nitroacetophenone with ammonium carbonate and formic acid.

A disadvantage of the processes mentioned is the high reaction temperatures to which the nitro compounds used are exposed, which can lead to considerable safety risks, especially on the industrial scale.

As an alternative to the reduction with formic acid, processes are also known in which the reduction of nitro-substituted acetophenones is carried out with sodium cyanoborohydride. J. L. Kelley, E. W. McLean, R. M. Ferris, J. L. Howard, J. Med. Chem. 1990, 33, 1910-1914 describe, for example, the preparation of 1-(3-nitrophenyl)ethylamine by reacting 3-nitroacetophenone with ammonium acetate and sodium cyanoborohydride in methanol at 25° C.

However, the very long reaction times and the use of the expensive and poisonous sodium cyanoborohydride reducing agent, make this process too industrially unattractive.

Another means of preparing nitro-substituted 1-phenylethylamine is the reduction of the nitro-substituted 1-azido-1-phenylethanes with triphenylphosphine, as described in WO-A 00/56331. However, the use of azides for the industrial synthesis is forbidden for safety reasons.

There is therefore still a need to provide a process by which nitro-substituted, enantiomerically enriched 1-phenylethylamines are obtainable in a simple and efficient manner.

### SUMMARY OF THE INVENTION

A process has now been found for preparing enantiomerically enriched compounds of the formula (I) 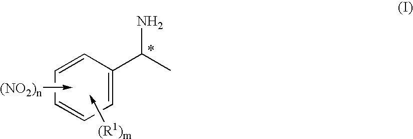
in which
* marks a stereogenic carbon atom,
n is 1, 2 or 3,
m is an integer in the range from 0 to (5-n) and
R¹ is in each case independently selected from the radicals: C₁-C₁₂-alkyl, C₁-C₁₋₂-alkoxy, C₁-C₁₂-alkylthio, C₅-C₁₄-aryl, C₅-C₁₄-aryloxy, C₆-C₁₅-arylalkyl, C₆-C₁₅-arylalkoxy, chlorine, fluorine, cyano, free or protected formyl, free or protected hydroxyl, free or protected mercapto, C₁-C₁₂-haloalkyl, C₁-C₁₂-haloalkylthio, C₁-C₁₂-haloalkoxy and radicals of the formulae (IIa) and (IIb)
   A-B-D-E (IIa)
   A-E (IIb)
   in which
A is absent or is a C₁-C₈-alkylene, C₁-C₈-alkenylene or C₁-C₈-haloalkylene radical and
B is absent or oxygen, sulphur or NR² where
R² is hydrogen, C₁-C₈-alkyl, C₆-C₁₅-arylalkyl or C₅-C₁₄-aryl and
D is a carbonyl group and
E is OR³ or N(R⁴)₂
   where
   R³ is C₁-C₈-alkyl, C₆-C₁₅-arylalkyl or C₅-C₁₄-aryl and
   R⁴ is in each case independently hydrogen, C₁-C₈-alkyl, C₆-C₁₅-arylalkyl or C₆-C₁₄-aryl, or N(R⁴)₂ together is a cyclic amino radical having 4 to 12 carbon atoms,
which is characterized in that,
   in a step A),
compounds of the formula (III) 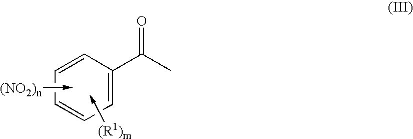
are reacted with compounds of the formula (IVa) or (IVb)
R⁵O—NH₂ (IVa)
R⁵O—NH₂.HX (IVb)
   in which
R⁵ is hydrogen or C₁-C₁₋₂-alkyl and
X is an anion
to initially give compounds of the formula (V) 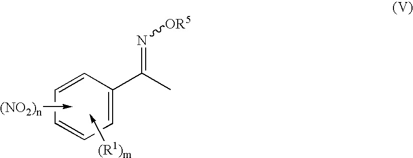
and,
   in a step B),
the compounds of the formula (V)
are reduced
   in an organic solvent
   with a complex borohydride and
   an acid
to racemic compounds of the formula (Ia) 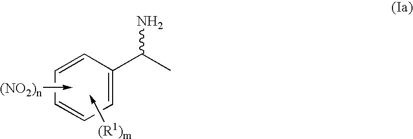
and,
in a step C),
   the racemic compounds of the formula (Ia) are enantiomerically enriched with the aid of enantiomerically enriched acids.

Optionally, as a further step D),
the enantiomerically enriched compounds of the formula (I) can be converted to the enantiomerically enriched ammonium salts of the formula (Ib).

In formula (Ib) 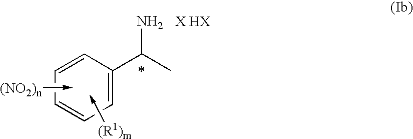
X is a halide or a sulphonate.

### DETAILED DESCRIPTION OF THE INVENTION

Preferred compounds of the formula (I) are those in which
n is 1,
m is 0 or 1 and
R¹ is in each case independently selected from the radicals: C₁-C₄-alkyl, C₁-C₄-alkoxy, C₁-C₄-alkylthio, C₅-C₁₄-aryl, C₅-C₁₄-aryloxy, C₆-C₁₅-arylalkyl, C₆-C₁₅-arylalkoxy, chlorine, fluorine, cyano, free or protected formyl, free or protected hydroxyl, free or protected mercapto and C₁-C₄-haloalkyl.

A particularly preferred compound of the formula (I) is 1-(3-nitrophenyl)ethylamine, and even greater preference is given to the (S)-enantiomer.

The same areas of preference apply correspondingly to compounds of the formula (Ib). Particularly preferred compounds of the formula (Ib) are (R)- and (S)-1-(3-nitrophenyl)ethylamine hydrochloride, and even greater preference is given to the (S)-enantiomer.

The enantiomerically enriched compounds of the formula (Ib) are likewise encompassed by the invention.

The reaction of the compounds of the formula (III) with the compounds of the formula (IVa) or (IVb) to give compounds of the formula (V) is preferably effected in an organic solvent, especially aliphatic alcohols. Particular preference is given to ethanol.

Preferred compounds of the formula (UVa) are: hydroxylamine and O-methylhydroxylamine, and particular preference is given to O-methylhydroxylamine.

Preferred compounds of the formula (IVb) are: hydroxylamine hydrochloride and O-methylhydroxylamine hydrochloride, and particular preference is given to O-methylhydroxylamine hydrochloride.

The reaction temperature in step A) may be, for example, 0° C. up to the boiling point of the solvent used; preference is given to a reaction at 10 to 100° C., particular preference to a reaction at 70 to 80° C.

The reduction in step B) takes place in the presence of
organic solvent
with a complex borohydride and
an acid.

Preferred organic solvents for step B) are ethers such as alkylene glycol alkyl ethers such as dipropylene glycol monomethyl ether, diethylene glycol monobutyl ether, diethyl ether, dioxane, tetrahydrofuran and tert-butyl methyl ether, optionally in a mixture with aromatic and/or aliphatic hydrocarbons.

Preferred complex borohydrides are those of the formula (VI)
Met[BH_{q}R⁶_{(4-q)}]ₚ (VI)
in which
Met is a mono- or divalent metal such as preferably zinc, lithium, sodium or potassium and
R⁶ is C₁-C₈-alkyl and
q is 1, 2, 3 or 4, preferably 4 or 1 and
p is the valency of Met.

Particular preference is given to sodium borohydride.

Step B) also takes place in the presence of acid. In this case, the term acid includes Lewis acids, for example titanium halides, zirconium halides and boron halides, and also Brönsted acids, especially carboxylic acids, and also compounds which form Lewis or Brönsted acids on reaction with complex borohydrides, for example iodine or di(C₁-C₄)alkyl sulphates.

Particular preference is given to carboxylic acids such as trifluoroacetic acid and to boron halides such as boron trifluoride, and very particular preference is given to boron trifluoride.

In step B), racemic compounds of the formula (Ia) are obtained.

In a preferred embodiment, the workup in step B) is effected by admixing with an aqueous acid or water and acid at a pH of <5 (based on room temperature) and extracting the aqueous solution with an organic solvent.

The acids used may be inorganic or organic acids, preferably inorganic acids, more preferably hydrochloric acid.

The extractants used may be organic solvents. Preference is given to alkyl ethers, aryl ethers, aliphatic hydrocarbons, aromatic hydrocarbons and halogenated hydrocarbons.

Particular preference is given to alkyl ethers, aryl ethers and halogenated hydrocarbons. Very particular preference is given to dichloromethane.

The compound of the formula (Ia) can then be released from the aqueous solution, for example, by admixing with a base to a pH of >8 and extracting with an organic solvent.

The bases used may be, for example and with preference, alkali metal or alkaline earth metal hydroxides and carbonates, or organic bases. Preference is given to alkali metal or alkaline earth metal hydroxides. Particular preference is given to sodium hydroxide.

The compound of the formula (Ia) can be extracted with the aid of an organic solvent. Preference is given to alkyl ethers, aryl ethers, aliphatic hydrocarbons, aromatic hydrocarbons and halogenated hydrocarbons. Particular preference is given to alkyl ethers, aryl ethers and halogenated hydrocarbons. Very particular preference is given to dichloromethane.

In step C), enantiomeric enrichment is effected with the aid of enantiomerically enriched acids.

Preference is given to enantiomerically enriched carboxylic or sulphonic acid, particular preference to camphorsulphonic acid.

In the context of this invention, the term “enantiomerically enriched carboxylic or sulphonic acids” refers to the particular enantiomerically pure carboxylic or sulphonic acids or to mixtures of the particular enantiomers in which one enantiomer is present in an enantiomeric excess, also referred to hereinbelow as ee, in comparison to the other enantiomer. This ee value is preferably 85 to 100%, more preferably 95 to 100% and most preferably 98 to 100%.

In all other contexts, the term “enantiomerically enriched” means enantiomerically pure substances (ee >99.5%) or mixtures of the particular enantiomers in which one enantiomer is present in an enantiomeric excess in comparison to the other enantiomer, and this ee value is preferably 10 to 100%, more preferably 50 to 100% and most preferably 70 to 100%.

In step C), the diastereomeric compounds of the formula (Ic) are then initially obtained 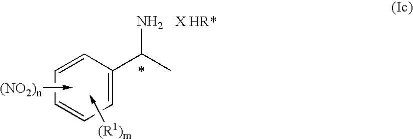
in which R* is the anion of an enantiomerically enriched organic acid.

As a consequence of their different physical data, the diastereomeric compounds of the formula (Ic) can be fractionally crystallized.

The compounds of the formula (Ic) in diastereomerically enriched form are likewise encompassed by the invention as important intermediates.

The nitro-substituted, enantiomerically enriched 1-phenylethylamines of the formula (I) can be released from the diastereomerically enriched compounds of the formula (Ic) by admixing with base and subsequently extracting with organic solvent, and the same areas of preference specified under B) apply for the term base and organic solvent.

In order to prevent oxidation of the free compounds of the formula (I), they can preferably subsequently be converted in step D) to compounds of the formula (Ib).

In the inventive manner, enantiomerically enriched, nitro-substituted 1-phenylethylamines of the formula (I) or the corresponding ammonium salts of the formula (Ib) can be obtained in high yields and ee values of up to 99.7.

The inventive nitro-substituted, enantiomerically enriched 1-phenylethylamines are especially suitable in a process for preparing active pharmaceutical ingredients, for example IMPDH inhibitors (see also WO-A 00/56331).

### EXAMPLES

### Example 1

### Preparation of 3-nitro-N-methoxyiminoacetophenone

240.0 g (1.453 mol) of 3-nitroacetophenone are suspended at room temperature in 1200 ml of ethanol. A solution of 132.0 g of O-methylhydroxylamine hydrochloride in 120 ml of water is added dropwise in 45 min to the suspension. The reaction mixture is heated to boiling and stirred at this temperature for 4 h. The suspension is subsequently hot-filtered. The filtrate is cooled to room temperature over a period of 12 h. The colourless solid which precipitates out in this time is filtered off with suction using a frit and washed three times with 100 ml of ethanol each time. The product is dried at 50° C. and a pressure of 100 mbar over a period of 2 h. 235.3 g (83.2% of theory) of a pale yellow solid are obtained.

### Example 2

### Preparation of 1-(3-nitrophenyl)ethylamine

a) Activation with Acetic Acid

100 g of dry tetrahydrofuran are initially charged under an argon atmosphere. 3.78 g (0.10 mol) of sodium borohydride are added to the solvent. The suspension is stirred at room temperature for 15 min. Subsequently, 6.0 g (0.10 mol) of acetic acid are added dropwise at a temperature of 20° C., in the course of which vigorous gas evolution sets in. Over a period of 20 min, a solution of 4.86 g (0.025 mol) of 3-nitro-N-methoxyiminoacetophenone in 40 ml of tetrahydrofuran is then added. The reaction mixture is stirred at room temperature for 2 h and subsequently heated to reflux for 2 h. Afterwards, the mixture is cooled to 110° C. and 60 ml of water are added dropwise with stirring at this temperature. The solvent is removed on a rotary evaporator. The residue is taken up in 150 ml of water and adjusted to pH 1 using 10 ml of conc. hydrochloric acid. The aqueous solution is washed with 50 ml of dichloromethane. Subsequently, the aqueous phase is adjusted to pH 9 using 4.5 ml of 45% sodium hydroxide solution and extracted twice with 25 ml each time of dichloromethane. The combined organic phases are dried over sodium sulphate. The solvent is removed on a rotary evaporator. 0.48 g (7.5% of theory) of the product is obtained as an orange oil.

b) Activation with Trifluoroacetic Acid

500 g of dry tetrahydrofuran are initially charged under a nitrogen atmosphere. 37.8 g (1.00 mol) of sodium borohydride are added to the solvent. The suspension is stirred at room temperature for 15 min. Subsequently, 114 g (1.00 mol) of trifluoroacetic acid are added dropwise at a temperature of 20° C., in the course of which vigorous gas evolution sets in. Over a period of 45 min, a solution of 48.6 g (0.25 mol) of 3-nitro-N-methoxyiminoacetophenone in 200 ml of dry tetrahydrofuran is then added. The reaction mixture is stirred at room temperature for 2 h and subsequently heated to 50° C. for 2 h. Afterwards, the mixture at 50° C. is metered over a period of 35 minutes into 400 ml of water with ice cooling. The reaction mixture is subsequently heated to 50° C. and stirred at this temperature for 2 h. Afterwards, the mixture is cooled to room temperature and adjusted to pH 1 using 130 ml of conc. hydrochloric acid. The solvent is distilled off on a rotary evaporator up to a bottom temperature of 50° C. at a pressure of 100 mbar. 500 ml of toluene are added to the residue. The reaction mixture is filtered. The organic phase is removed. The aqueous phase is washed with 500 ml of toluene. The organic phases are discarded. The aqueous phase is adjusted to pH 10 using 60 ml of 45% sodium hydroxide solution and extracted twice with 500 ml each time of toluene. The solvent of the combined organic phases is removed on a rotary evaporator. 27.6 g (58.2% of theory) of the product are obtained as an orange oil.

c) Activation with Boron Trifluoride-Diethyl Ether Complex

106 g of dry tetrahydrofuran are initially charged under a nitrogen atmosphere. 61.4 g (1.54 mol) of sodium borohydride are added to the solvent. The suspension is stirred at room temperature for 15 min. Over a period of 35 min, a solution of 100.0 g (0.51 mol) of 3-nitro-N-methoxyiminoacetophenone and 501 g of dry tetrahydrofuran in 228 g of toluene is then added. Subsequently, 219 g (1.54 mol) of boron trifluoride-diethyl ether complex is added dropwise at a temperature of 20° C. within 30 min, in the course of which vigorous gas evolution sets in. The reaction mixture is stirred at room temperature for 5 h and subsequently heated to 40° C. for 2 h. Afterwards, the mixture is metered over a period of 4 h into 1200 ml of water with ice cooling. The reaction mixture is subsequently stirred at 20° C. for 10 min. Afterwards, the mixture is adjusted to pH 1 using 107 ml of conc. hydrochloric acid, heated to 50° C. and stirred for 2 h. The phases are separated. The organic phase is discarded. The aqueous phase is washed with 500 ml of dichloromethane. The organic phase is discarded. The aqueous phase is filtered, adjusted to pH 10 using 140 ml of 45% sodium hydroxide solution and extracted three times with 500 ml each time of dichloromethane. The solvent of the combined organic phases is removed on a rotary evaporator. 66.3 g (78.3% of theory) of the product are obtained as an orange oil.

### Example 3

### Preparation of (S)-1-(3-nitrophenyl)ethylamine

a) Crystallization with L-(+)-tartaric Acid

0.75 g (5.0 mmol) of L-(+)-tartaric acid is dissolved in 64 ml of methanol and the solution is heated to reflux. A solution of 1.0 g (6.0 mmol) of 1-(3-nitrophenyl)ethylamine from Example 2 in 7.1 ml of methanol is metered in over a period of 5 min and the mixture is subsequently stirred under reflux for 15 min.

The solution is cooled to 50° C. over a period of 40 min and subsequently to 35° C. over a period of 1.5 h. The solution is then stored at 5° C. for 4 days. The solid which precipitates out in this time is filtered off. 0.5 g of a beige solid is obtained. This is suspended in 10 ml of water and adjusted to pH 10 using 0.9 ml of 10% sodium hydroxide solution. The solution is extracted twice with 30 ml each time of dichloromethane. The solvent of the combined organic phases is removed on a rotary evaporator. 0.3 g (30% of theory) of (S)-1-(3-nitrophenyl)ethylamine is obtained in the form of a brown oil. The ee value is 70%.

b) Crystallization with (+)-camphorsulphonic Acid

226.7 g (1.364 mol) of 1-(3-nitrophenyl)ethylamine from Example 2 are added at 22° C. to 2830 ml of water. Over a period of 5 min, 301.0 g (1.296 mol) of (+)-camphorsulphonic acid are added. In the course of this, the temperature rises to 30° C. The reaction mixture is heated to 60° C. and stirred at this temperature for 10 min. The mixture is then cooled to 10° C. over a period of 18 h. The precipitated solid is filtered off with suction and washed four times with 100 ml each time of water. 224.5 g of a beige product are obtained. The ee value is 92.6%.

222.0 g of the resulting solid are suspended in 500 ml of water. The suspension is heated to 95° C. and stirred at this temperature for 10 min. The solution is cooled to 10° C. over a period of 18 h. The precipitated solid is filtered off with suction and washed three times with 100 ml each time of water. The solid is dried at a pressure of 100 mbar for 1 h. 128.7 g (overall 23.7% of theory) of a beige solid are obtained. The ee value is 99.7%.

### Example 4

### Preparation of (S)-1-3-nitrophenyl)ethylamine Hydrochloride

126.5 g (317 mmol) of the thus obtained (S)-1-(3-nitrophenyl)ethylamine (+)-camphorsulphonate are dissolved in 200 ml of 2N sodium hydroxide solution. 100 ml of water are added to the solution. The phases are separated. The aqueous phase is extracted twice with 100 ml of dichloromethane. The solvent of the combined organic phases is removed on a rotary evaporator. 100 ml of dichloromethane and 5 g of activated carbon are added to the residue. The solution is filtered through Celite. The filter residue is washed with 50 ml of dichloromethane.

Hydrogen chloride gas is passed through the filtrate over 20 min. Subsequently, nitrogen is passed through the suspension for 3 min. The solid is filtered off with suction and washed twice with 50 ml each time of dichloromethane. After the solid has been dried at 40° C. and a pressure of 100 mbar, 53.0 g (100% of theory) of a beige product are obtained. The ee value is 99.6%.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. Process for preparing enantiomerically enriched compounds of the formula (I) 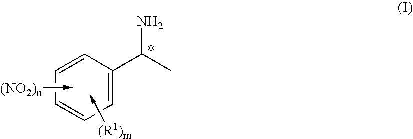
in which
* marks a stereogenic carbon atom,
n is 1, 2 or 3,
m is an integer in the range from 0 to (5-n) and
R¹ is in each case independently selected from the radicals: C₁-C₁₂-alkyl, C₁-C₁₂-alkoxy, C₁-C₁₋₂-alkylthio, C₅-C₁₄-aryl, C₅-C₁₄-aryloxy, C₆-C₁₅-arylalkyl, C₆-C₁₅-arylalkoxy, chlorine, fluorine, cyano, free or protected formyl, free or protected hydroxyl, free or protected mercapto, C₁-C₁₂-haloalkyl, C₁-C₁₂-haloalkylthio, C₁-C₁₂-haloalkoxy and radicals of the formulae (IIa) and (IIb)
A-B-D-E (IIa)
A-E (IIb)
in which
A is absent or is a C₁-C₈-alkylene, C₁-C₈-alkenylene or C₁-C₈-haloalkylene radical and
B is absent or oxygen, sulphur or NR² where
R² is hydrogen, C₁-C₈-alkyl, C₆-C₁₅-arylalkyl or C₅-C₁₄-aryl and
D is a carbonyl group and
E is OR³ or N(R⁴)₂
where
R³ is C₁-C₈-alkyl, C₆-C₁₅-arylalkyl or C₅-C₁₄-aryl and
R⁴ is in each case independently hydrogen, C₁-C₈-alkyl, C₆-C₁₅-arylalkyl or C₆-C₁₄-aryl, or N(R⁴)₂ together is a cyclic amino radical having 4 to 12 carbon atoms,
comprising,
* in a step A), reacting
compounds of the formula (III) 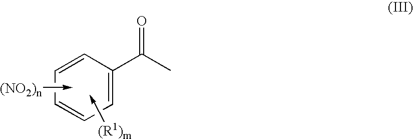
with compounds of the formula (IVa) or (IVb)
R⁵O—NH₂ (IVa)
R⁵O—NH₂.HX (IVb)
in which
R⁵ is hydrogen or C₁-C₁₂-alkyl and
X is an anion
to initially give compounds of the formula (V) 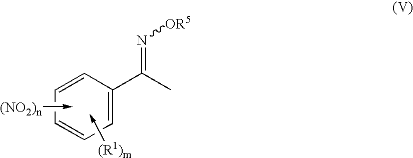
and,
in a step B), reducing
the compounds of the formula (V)
in an organic solvent
with a complex borohydride and
an acid
to racemic compounds of the formula (Ia) 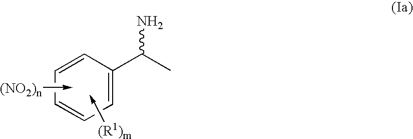
and,
in a step C), enriching
the racemic compounds of the formula (Ia) enantiomerically with the aid of enantiomerically enriched acids.

2. Process according to claim 1, further comprising step D), of converting the enantiomerically enriched compounds of the formula (I) to enantiomerically enriched ammonium salts of the formula (Ib) 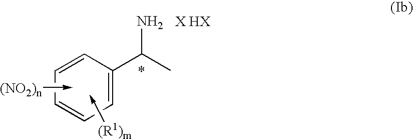
in which X is a halide or a sulphonate.

3. Process according to claim 1, wherein
n is 1,
m is 0 or 1 and
R¹ is in each case independently selected from the radicals: C₁-C₄-alkyl, C₁-C₄-alkoxy, C₁-C₄-alkylthio, C₅-C₁₄-aryl, C₅-C₁₄-aryloxy, C₆-C₁₅-arylalkyl, C₆-C₁₅-arylalkoxy, chlorine, fluorine, cyano, free or protected formyl, free or protected hydroxyl, free or protected mercapto and C₁-C₄-haloalkyl.

4. Process according to claim 1, wherein 1-(3-nitrophenyl)ethylamine or 1-(3-nitrophenyl)ethylamine hydrochloride are prepared.

5. Process according to claim 1, wherein the complex borohydrides used in step B) are those of the formula (VI)
Met[BH_{q}R⁶_{(4-q)}]ₚ (VI)
in which
Met is a mono- or divalent metal such as preferably zinc, lithium, sodium or potassium and
R⁶ is C₁-C₈-alkyl and
q is 1, 2, 3 or 4, preferably 4 or 1 and
p is the valency of Met.

6. Process according to claim 1, wherein the acids used in step B) are Lewis acids, Brönsted acids and compounds which form Lewis or Brönsted acids on reaction with complex borohydrides.

7. Process according to claim 1, wherein camphorsulphonic acid is used in step C).

8. Diastereomerically enriched compounds of the formula (Ic) 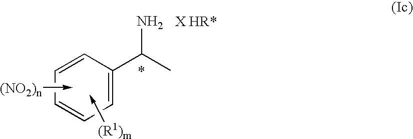
in which n, m and R¹ are each as defined in claim 1 and R* is the anion of an enantiomerically enriched organic acid.

9. (S)-1-(3-Nitrophenyl)ethylammonium camphorsulphonate.

10. Enantiomerically enriched compounds of the formula (Ib) 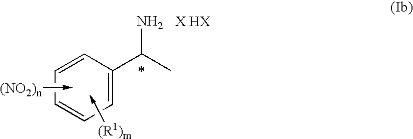
in which n, m and R¹ are each as defined in claim 1 and X is a halide.

11. (S)-1-(3-Nitrophenyl)ethylammonium chloride.

12. A process for preparing pharmaceutical ingredients comprising incorporating compounds according to claim 8.

13. A process for preparing pharmaceutical ingredients comprising incorporating compounds according to claim 9.

14. A process for preparing pharmaceutical ingredients comprising incorporating compounds according to claim 10.

15. A process for preparing pharmaceutical ingredients comprising incorporating compounds according to claim 11.
